(19) United States
(12) Patent Application Publication
(10) Pub. No.: US 2023/0041051 A1
(43) Pub. Date: Feb. 9, 2023

(54) METHODS AND SYSTEMS FOR PREDICTING AND PREVENTING FREQUENT PATIENT READMISSION

(71) Applicant: KONINKLIJKE PHILIPS N.V., EINDHOVEN (NL)

(72) Inventors: Luoluo Liu, Cambridge, MA (US); Eran Simhon, Cambridge, MA (US)

(21) Appl. No.: 17/879,839

(22) Filed: Aug. 3, 2022

Related U.S. Application Data
(60) Provisional application No. 63/229,119, filed on Aug. 4, 2021.

(51) Int. Cl. G16H 50/30

(52) U.S. Cl. CPC G16H 50/30

(57) ABSTRACT

A method for presenting a patient frequent readmission recommendation, comprising: (i) receiving patient information comprising a plurality of demographic and/or medical features; (ii) extracting the features from the information; (iii) analyzing the features to determine whether the patient is a frequent readmission patient or is at risk of being a frequent readmission patient; (iv) estimating, if the patient is determined to be a frequent readmission patient, whether the frequent readmission is due to a medical condition and/or a socioeconomic condition, or predicting a frequent readmission risk level if the patient is determined to be at risk of being a frequent readmission patient; (v) generating a recommendation based at least in part on the estimated condition or the frequent readmission risk level, wherein the recommendation comprises a medical intervention and/or a socio-behavioral intervention; and (vi) providing (180) the recommendation via a user interface.

### FIELD OF THE DISCLOSURE

The present disclosure is directed generally to methods and systems for generating and presenting patient frequent readmission recommendations using a patient frequent readmission risk system.

### BACKGROUND

Patient readmission occurs when a patient is readmitted to a hospital or other care facility within a certain period of time (e.g., days or weeks) after having been discharged, usually for treatment of the same or related condition. Readmission rates can be especially high for certain conditions and for certain people. However, readmission can be unfavorable for several reasons. For example, high readmission frequency can have significant financial consequences for the care facility. Frequent readmission is becoming an increasingly important problem in the United States, due in large part to resource utilization and financial incentives and/or penalties from payers discouraging preventable readmissions.

Indeed, high utilizers or superusers of medical care, also known as “frequent flyers,” and “recurrent patients,” constitute a very small percentage of medical care recipients but impose a disproportionately high utilization of resources and therefore create undue burden on healthcare facilities and professionals due to their elevated resource use. Studies have suggested that 1% of Americans account for approximately 22% of healthcare spending, and that 5% of Americans account for approximately 50% of all healthcare spending. Despite this, there is no standardized criteria for identifying a patient as a high utilizer or superuser. Further, it has been suggested that flagging a patient as a high utilizer or superuser can have a negative influence on care, such as eliciting implicit negative bias from healthcare professionals.

Patients can be a high utilizer or superuser for medical reasons and/or for socioeconomic reasons. For example, common medical reasons include chronic obstructive pulmonary disease (COPD), heart failure, kidney failure, neurosis, among other reasons. Common social economic factors are lack of insurance, poor economic status, substance abuse, mental illness, among other reasons.

While high utilizers or superusers of medical care utilize a disproportionate amount of healthcare resources, these frequent visits can be prevented if there are early warnings and treatments to ameliorate the medical and/or socioeconomic risk factors. Therefore, identifying superusers leads to interventions that can improve both operational efficiency and patient outcomes while significantly reducing medical care expenditures.

### SUMMARY OF THE DISCLOSURE

Accordingly, there is a continued need for methods and systems that predict frequent readmission visits or risk for a patient and that provide recommendations to reduce visits or reduce risk. Various embodiments and implementations herein are directed to a method and system configured to generate and present a patient frequent readmission recommendation using a patient frequent readmission risk system. The system receives patient information comprising demographic and/or medical features, and the system extracts the plurality of demographic and/or medical features from the received information. A trained model of the system analyses the extracted demographic and/or medical features to determine whether the patient is a frequent readmission patient or is at risk of being a frequent readmission patient. If the patient is determined to be a frequent readmission patient, the system estimated whether the frequent readmission is due to a medical condition and/or due to a socioeconomic condition. If the patient is determined to be at risk of being a frequent readmission patient, the system predicts a frequent readmission risk level which includes an identification of a medical factor and/or socioeconomic factor impacting the frequent readmission risk. Based at least in part on the estimated condition or the predicted frequent readmission risk level, the system generates a recommendation. The recommendation comprises a medical intervention if the frequent readmission is estimated to be due to a medical condition or if the risk of being a frequent readmission patient is based on a medical factor, or comprises a socio-behavioral intervention if the frequent readmission is estimated to be due to a socioeconomic condition or if the risk of being a frequent readmission patient is based on a socioeconomic factor. The system provides the recommendation to a user of the patient frequent readmission risk system using a user interface.

Generally, in one aspect, a method for generating and presenting a patient frequent readmission recommendation using a patient frequent readmission risk system is provided. The method includes: (i) receiving, at the patient frequent readmission risk system, patient information, wherein the patient information comprises a plurality of demographic and/or medical features; (ii) extracting, by a processor of the patient frequent readmission risk system, the plurality of demographic and/or medical features from the received information; (iii) analyzing, by a trained model of the patient frequent readmission risk system, the extracted demographic and/or medical features to determine whether the patient is a frequent readmission patient or is at risk of being a frequent readmission patient; (iv) estimating, if the patient is determined to be a frequent readmission patient, whether the frequent readmission is due to a medical condition and/or due to a socioeconomic condition, or predicting a frequent readmission risk level if the patient is determined to be at risk of being a frequent readmission patient, wherein the predicted frequent readmission risk level further comprises an identification of a medical factor and/or socioeconomic factor impacting the frequent readmission risk; (v) generating a recommendation based at least in part on the estimated condition or the frequent readmission risk level, wherein the recommendation comprises a medical intervention if the frequent readmission is estimated to be due to a medical condition or if the risk of being a frequent readmission patient is based on a medical factor, and wherein the recommendation comprises a socio-behavioral intervention if the frequent readmission is estimated to be due to a socioeconomic condition or if the risk of being a frequent readmission patient is based on a socioeconomic factor; and (vi) providing the recommendation via a user interface of the patient frequent readmission risk system.

According to an embodiment, the method further includes implementing the provided medical intervention or socio-behavioral intervention.

According to an embodiment, the provided recommendation comprises a visualization of the patient's visitation frequency, frequent readmission risk level, recommendation, medical condition, and/or socioeconomic condition.

According to an embodiment, at least some of the patient information is received from a patient home monitoring device. According to an embodiment, at least some of the patient information is received from an electronic medical records database or system.

According to an embodiment, the medical features comprise information about patient diagnosis, patient treatment, and/or or a number of healthcare facility admissions during a previous predetermined time period.

According to an embodiment, a patient is determined to be a frequent readmission patient if the patient has been admitted to a healthcare facility at least a predetermined number of times during a previous predetermined time period.

According to an embodiment, the method further includes training the model of the patient frequent readmission risk system using historical patient data.

According to a second embodiment is a patient frequent readmission risk system configured to generate and present a patient frequent readmission recommendation. The system includes: a trained patient frequent readmission risk model configured to determine, based on one or more demographic and/or medical features, whether a patient is a frequent readmission patient or is at risk of being a frequent readmission patient; a processor configured to: (i) receive patient information comprising a plurality of demographic and/or medical features; (ii) extract the plurality of demographic and/or medical features from the received information; (iii) analyze, by the trained patient frequent readmission risk model, the extracted demographic and/or medical features to determine whether the patient is a frequent readmission patient or is at risk of being a frequent readmission patient; (iv) estimate, if the patient is determined to be a frequent readmission patient, whether the frequent readmission is due to a medical condition and/or due to a socioeconomic condition, or predict a frequent readmission risk level if the patient is determined to be at risk of being a frequent readmission patient, wherein the predicted frequent readmission risk level further comprises an identification of a medical factor and/or socioeconomic factor impacting the frequent readmission risk; (v) generate a recommendation based at least in part on the estimated condition or the frequent readmission risk level, wherein the recommendation comprises a medical intervention if the frequent readmission is estimated to be due to a medical condition or if the risk of being a frequent readmission patient is based on a medical factor, and wherein the recommendation comprises a socio-behavioral intervention if the frequent readmission is estimated to be due to a socioeconomic condition or if the risk of being a frequent readmission patient is based on a socioeconomic factor; and a user interface configured to present to a user the generated recommendation.

According to an embodiment, the processor is further configured to receive an input from a user regarding an implementation of the provided medical intervention or socio-behavioral intervention.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 is a flowchart of a method for generating and presenting a patient frequent readmission recommendation, in accordance with an embodiment.

FIG. 2 is a schematic representation of a patient frequent readmission risk system, in accordance with an embodiment.

FIG. 3 is a flowchart of a method for training a patient frequent readmission risk system, in accordance with an embodiment.

FIG. 4 is a flowchart of a method for training a patient frequent readmission risk system, in accordance with an embodiment.

FIG. 5 is a schematic representation of output of a patient frequent readmission risk model, in accordance with an embodiment.

FIG. 6 is a flowchart of a method for generating and presenting a patient frequent readmission recommendation, in accordance with an embodiment.

FIG. 7 is a flowchart of decision flow of recommendations or suggestions for a clinician and/or a user of the patient frequent readmission risk system, in accordance with an embodiment.

FIG. 8 is a schematic representation of an output of a patient frequent readmission risk system, in accordance with an embodiment.

### DETAILED DESCRIPTION OF EMBODIMENTS

The present disclosure describes various embodiments of a system and method configured to generate and present a patient frequent readmission recommendation. More generally, Applicant has recognized and appreciated that it would be beneficial to provide a method and system to prevent disproportionate utilization of healthcare resources by superusers. Accordingly, a trained frequent readmission system analyzes and communicates superuser risks and recommendations to a clinician, which allows the clinician to make decisions about in-facility and/or post-discharge medical and/or social care. The trained patient frequent readmission risk system receives information about a patient, the information comprising demographic and/or medical features, and extracts the plurality of demographic and/or medical features from the received information. A trained model of the system analyses the extracted demographic and/or medical features to determine whether the patient is a frequent readmission patient or is at risk of being a frequent readmission patient. If the patient is determined to be a frequent readmission patient, the system estimated whether the frequent readmission is due to a medical condition and/or due to a socioeconomic condition. If the patient is determined to be at risk of being a frequent readmission patient, the system predicts a frequent readmission risk level which includes an identification of a medical factor and/or socioeconomic factor impacting the frequent readmission risk. Based at least in part on the estimated condition or the predicted frequent readmission risk level, the system generates a recommendation. The recommendation comprises a medical intervention if the frequent readmission is estimated to be due to a medical condition or if the risk of being a frequent readmission patient is based on a medical factor, or comprises a socio-behavioral intervention if the frequent readmission is estimated to be due to a socioeconomic condition or if the risk of being a frequent readmission patient is based on a socioeconomic factor. The system provides the recommendation to a user of the patient frequent readmission risk system using a user interface.

According to an embodiment, the systems and methods described or otherwise envisioned herein can be utilized to generate rule-based algorithms to identify and label high utilizers or superusers, or to identify and label visits by utilizers or superusers. According to another embodiment, the systems and methods described or otherwise envisioned herein can be utilized as a tool for chief operating officers, patient flow coordinators, charge nurses, house supervisors, emergency department nurses and managers, and many other healthcare professionals, to etc. to monitor and analyze frequent visits. According to another embodiment, the systems and methods described or otherwise envisioned herein can be utilized to model utilizers or superusers and explore contributing factors while providing guidance and recommendations to prevent or reduce visits by utilizers or superusers, thereby improving operational efficiency and patient outcomes.

According to an embodiment, the systems and methods described or otherwise envisioned herein can, in some non-limiting embodiments, be implemented as an element for a commercial product for patient analysis or monitoring, such as the Philips® Patient Flow Capacity Suite (PFCS) (available from Koninklijke Philips NV, the Netherlands), or any suitable patient or care facility system.

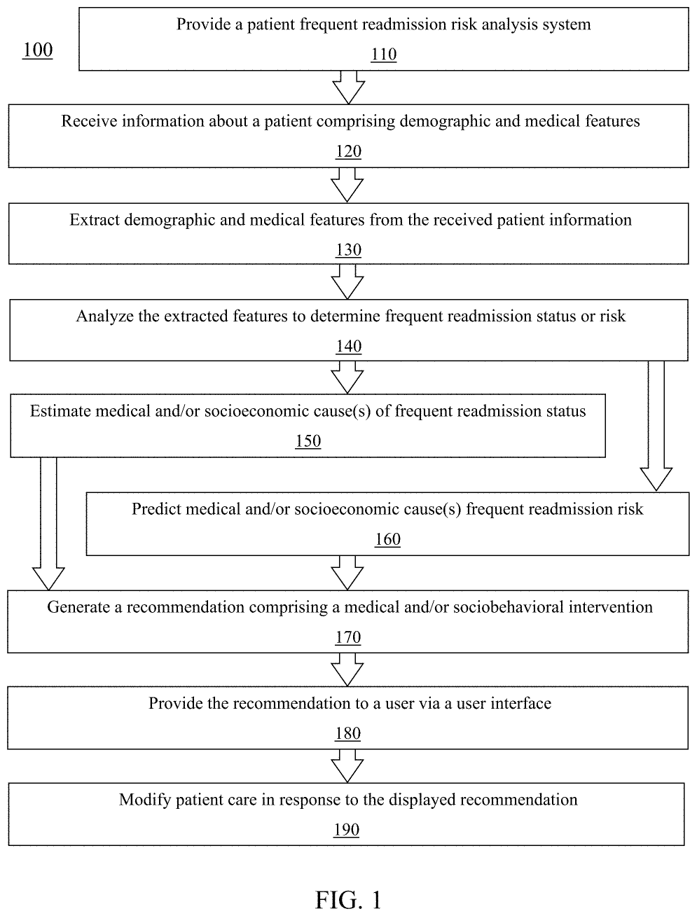

Referring to FIG. 1, in one embodiment, is a flowchart of a method 100 for generating and communicating a patient frequent readmission recommendation using a patient frequent readmission risk system. The methods described in connection with the figures are provided as examples only, and shall be understood not limit the scope of the disclosure. The patient frequent readmission risk system can be any of the systems described or otherwise envisioned herein. The patient frequent readmission risk system can be a single system or multiple different systems.

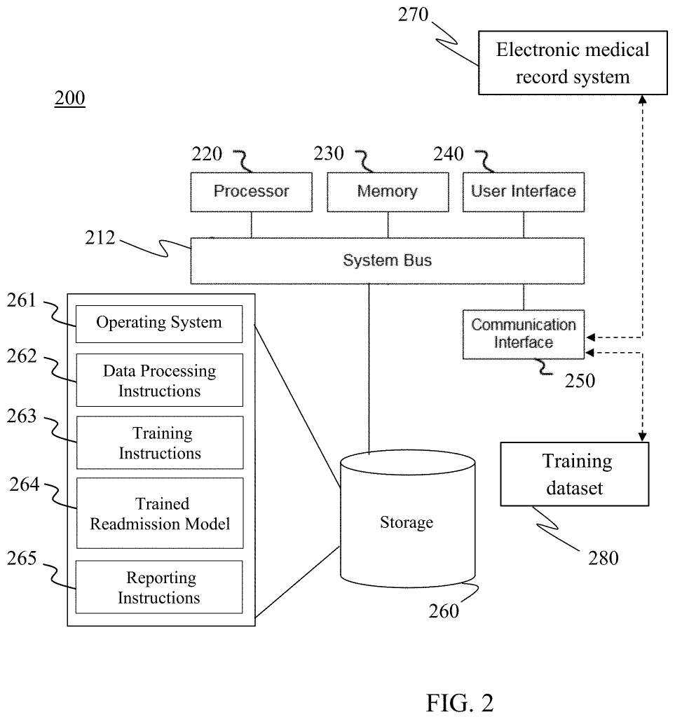

At step 110 of the method, a patient frequent readmission risk system is provided. Referring to an embodiment of a patient frequent readmission risk system 200 as depicted in FIG. 2, for example, the system comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the system 200 may be different and more complex than illustrated. Additionally, patient frequent readmission risk system 200 can be any of the systems described or otherwise envisioned herein. Other elements and components of patient frequent readmission risk system 200 are disclosed and/or envisioned elsewhere herein.

At step 120 of the method, the patient frequent readmission risk system receives information about a patient for which an analysis will be performed. According to an embodiment, the information comprises a plurality of features about the patient. Only some, or all, of the information about the patient and features may ultimately be utilized by the patient frequent readmission risk system. The plurality of features may comprise, for example, demographic and/or medical features about the patient. For example, demographic information or features may comprise age, gender, past healthcare facility visits or admissions, and other demographic information. Medical information or features may comprise vital sign information about the patient, including but not limited to physiologic vital signs such as heart rate, blood pressure, respiratory rate, apnea, SpO₂, invasive arterial pressure, noninvasive blood pressure, physiological measurements other than vital data such as physical observations, patient diagnosis or medication condition such as cardiac disease, psychological disorders, chronic obstructive pulmonary disease, and more, among many other types of medical information. Many other types of patient information are possible. Accordingly, the received information can be any information relevant to a patient frequent readmission risk analysis.

The patient frequent readmission risk system can receive patient information from a variety of different sources, including any source that comprises one or more patient features. According to an embodiment, the patient frequent readmission risk system is in communication with an electronic medical records database from which the patient information and one or more of the plurality of features may be obtained or received. The electronic medical records database may be a local or remote database and is in communication with the patient frequent readmission risk system 200. According to an embodiment, the patient frequent readmission risk system comprises an electronic medical record database or system 270 which is optionally in direct and/or indirect communication with system 200. According to another embodiment, the patient frequent readmission risk system may obtain or receive the plurality of features from equipment or a healthcare professional obtaining that information directly from the patient. According to another embodiment, the patient frequent readmission risk system may obtain or receive the plurality of features from home monitoring equipment, such as a wearable device, CPAP machine, or any other device utilized by a patient outside of a hospital or other direct healthcare facility.

According to an embodiment, the patient information includes patient demographics, clinical and operational information from multiple healthcare information systems, including electronic medical records (EMR), radiology information systems, cardiology information systems, lab information systems, and other systems. From these sources, the following types of information that are useful for risk estimation can be linked and collected: (i) patient demographic information (for example age, gender, race, height, weight, etc.); (ii) previous hospital visit operational information (for example time of appointment and visit, reason for visit, healthcare providers, etc.) within a previous time period such as a year although many other time periods are possible; (ii) current hospital visits clinical information (for example diagnosis, medical imaging and report, treatment, medication, etc.); (iii) medical history and family history; (iv) other information from other sources (for example geographic-based census information, socioeconomic information, personal survey) could also be leveraged to for analysis; and many other types or sources of patient information. According to an embodiment, the patient frequent readmission risk system may query an electronic medical record database or system, comprising fast healthcare interoperability resources (FHIR) for example, to obtain the patient information.

The patient information received by the patient frequent readmission risk system may be processed by the system according to methods for data handling and processing/preparation, including but not limited to the methods described or otherwise envisioned herein. The patient information received by the patient frequent readmission risk system may be utilized, before or after processing, immediately or may be stored in local or remote storage for use in further steps of the method.

At step 130 of the method, the patient frequent readmission risk system extracts demographic and/or medical features from the patient information received in step 120 of the method. The features can be any of the features described or otherwise envisioned herein. The system can be trained to identify and extract the patient features from the received patient information, using any of a wide variety of algorithms, methods, or systems for identifying and extracting patient data. The plurality of identified and extracted patient features may be utilized immediately or may be stored in local or remote storage for use in further steps of the method.

According to an embodiment, the patient information and/or patient features may undergo data processing at any stage. For example, values from an EMR database may be remapped, as it is common that there are values that corresponds to the same value however are recorded with small differences. For example, both “unspecified” and “unknown” mean the same thing. Accordingly, the system can utilize look-up tables for categorical variables and subgroup categories. As another example of data processing, data with missing admission timestamps can be removed or flagged. Since it is important to detect frequent visits using admission timestamps, data with missing admission timestamps can therefore be removed.

As another example of data processing, data can be imputed. Missing data can be imputed using existing information. As an example, an admission that is missing discharge time could be imputed by adding a shift from the admission timestamps sampling from an empirical length of stay distribution of the same admission type. For other features that are missing, the system can fill them with sample median or random samples from the empirical distribution of the same admission type. As another example of data processing, data can be binned. As an example, for continuous variable such as age, it is standard to use age bins rather than the exact age. It is especially common for an EMR dataset to add shifts to the age value for very old patients for the purpose of deidentification. This practice can be used to resolve inaccurate large ages and most importantly reduce the feature space size of the model. Age is one of the dominate factors for frequent visits, and therefore it is advantageous to use the actual age rather than age bins. However, age bins could be used for the reasons mentioned above. As another example of data processing, data can be normalized to be readied for input. This could include scaling continuous variables, hot-encode categorical variables, and other types of data normalization.

At step 140 of the method, a trained model of the patient frequent readmission risk system analyzes the extracted demographic and/or medical features to determine whether the patient is a frequent readmission patient or is at risk of being a frequent readmission patient. The trained model may be any module, algorithm, or machine learning method for analyzing input features and determining whether the patient is a frequent readmission patient or is at risk of being a frequent readmission patient.

According to an embodiment, the trained model analyzes data such as patient visits within a predetermined prior time period to determine whether the patient is a frequent readmission patient or whether this visit is a frequent readmission visit. For example, the model may comprise a rule-based method to identify frequent readmission patients based on one or more majority rules in the literature for inpatient visits and emergency department (ED) visits. The rule(s) can be based, for example, on one-year historical inpatient and ED visits counts and the time of the most recent admission. For example, 30-day readmission number or value is widely used and recognized by Centers of Medicare and Medicaid Services. As another example, the number of inpatient visits within a year is utilized in scientific literature as one of the common criteria for identifying frequent readmission patients. In addition, at least one literature search found that 41% of publications utilized the number of in patient versus admission to analyze frequent readmission patients. As another example, the number of emergency department visits within a year is adopted widely in the scientific literature. At least one literature search found that 72.5% of publications utilized the number emergency department visits to analyze frequent readmission patients, making this criterion the top one in defining frequent readmission. The second top criteria targeting high utilization is the number of inpatient admissions.

According to one embodiment, for the number of ED visits and inpatient visits within the previous year, a value of four (4) visits can be used as a threshold, although many other values are possible. This threshold number is taken from literature which, based on 100 literature articles, describes 180 criteria for marking frequent users. Among criteria using the number of ED visits, 41% used four as a threshold and, among the ones using the number of inpatient admissions, 44% used four as threshold. Moreover, for each module the thresholds can be set by default thresholds, which is also customizable by adding optional configuration parameters.

According to an embodiment, the trained model of the patient frequent readmission risk system can be trained according to a wide variety of methods and approaches. As one example, the model may comprise a neural network approach. According to an embodiment, using a neural network approach such as multi-layer perception (MLP) can learn representation better and can improve recall by at least 2%, with performance improvement 15% on the recall rate. Recent work called TabNet has shown promising results on benchmark tabular datasets, and is another example of an alternative network design for predicting frequent patient readmission risk. According to an embodiment, the model is validated using training on 80% training data and validated on the remaining 20% as testing data. As other examples, the model may comprise a decision tree approach like Xgboost, or statistic-based method such as association rules. The final model parameters can be selected based on performance matrices such as curve (AUC) and area under the precision-recall curve (AUCPR), among other approaches.

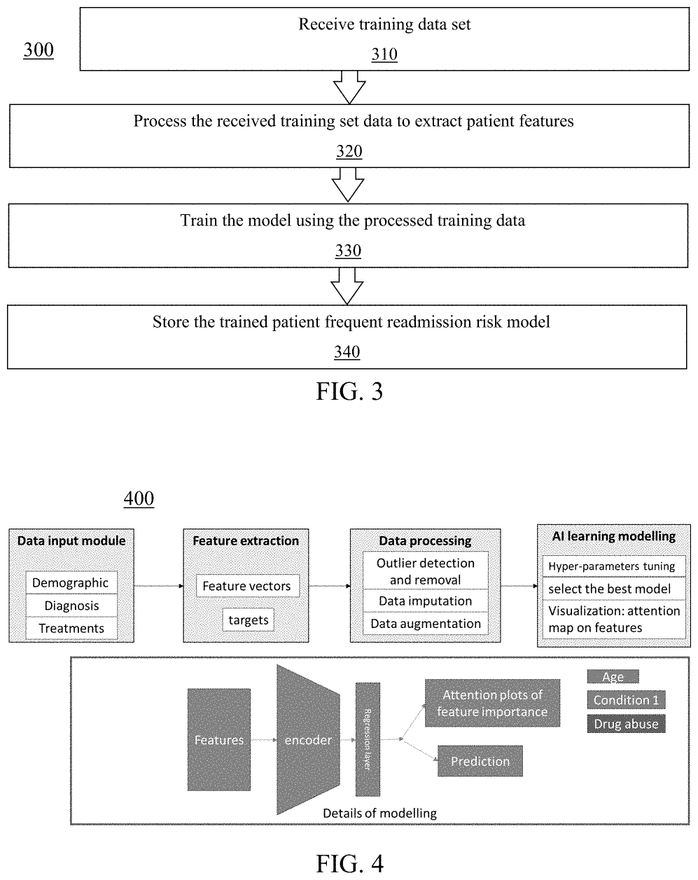

Referring to FIG. 3, in one embodiment, is a flowchart of a method 300 for training the patient frequent readmission risk model of the patient frequent readmission risk system. At step 310 of the method, the system receives a training data set comprising training data about a plurality of patients. The training data can comprise demographic and medical information about each of the patients, including but not limited to demographics, physiological measurements such as vital data, physical observations, and/or diagnosis, prior inpatient and emergency department visits, and many other types of information. As an example, the demographic and medical information can include detailed information on patient demographics such as age, gender, and more; diagnosis or medication condition such as cardiac disease, psychological disorders, chronic obstructive pulmonary disease, and more; physiologic vital signs, and more. Many other types of information are possible. According to an embodiment, the training data may also comprise an indication or information about one or more outcomes of each patient, including readmissions. According to an embodiment, the training data may comprise retrospective admission/discharge/transfer (ADT) data. The training data may be stored in and/or received from one or more databases. The database may be a local and/or remote database. For example, the patient frequent readmission risk system may comprise a database of training data.

According to an embodiment, the patient frequent readmission risk system may comprise a data pre-processor or similar component or algorithm configured to process the received training data. For example, the data pre-processor analyzes the training data to remove noise, bias, errors, and other potential issues. The data pre-processor may also analyze the input data to remove low quality data. Many other forms of data pre-processing or data point identification and/or extraction are possible.

At step 320 of the method, the system processes the received information to extract demographic and/or medical features about one or more of the plurality of patients. The extracted features may be any features which will be utilized to train the model, such as any frequent readmission prediction features that can or will be utilized by the trained algorithm for patient frequent readmission analysis or frequent readmission risk analysis for a future patient. Feature extraction can be accomplished by a variety of embodiments for feature identification, extraction, and/or processing, including any method for extracting features from a dataset. According to an embodiment, the extracted patient features may undergo data processing at any stage. For example, the extracted patient features may be processed using any of the data processing methods described or otherwise envisioned herein. The outcome of a feature processing step or module of the patient frequent readmission risk system is a set of features about a plurality of patients, which thus comprises a training data set that can be utilized to train the classifier.

At step 330 of the method, the system trains the machine learning algorithm, which will be the algorithm utilized in analyzing patient information as described or otherwise envisioned. The machine learning algorithm is trained using the extracted features according to known methods for training a machine learning algorithm. According to an embodiment, the algorithm is trained, using the processed training dataset, to analyze extracted demographic and/or medical features to determine whether a patient is a frequent readmission patient or is at risk of being a frequent readmission patient.

At step 340 of the method, the trained patient frequent readmission model is stored for future use. According to an embodiment, the model may be stored in local or remote storage for use in further steps of the method.

Referring to FIG. 4 is a flowchart of a method 400 for training the model of the patient frequent readmission risk system. According to an embodiment, the model contains an encoder and a decoder. Because the feature size in this problem is relatively small, it can be optionally recommended that the encoder comprise only a few layers, rather than a very deep structure. The encoder can be composed of fully connected layers, or gated layers with attention layers. From the encoder, latent variables are generated and feed to a decoder. In this case, a regression layer and a sigmoid function at the end to make the output vector between 0 and 1, for a proper output of probability. The model can be trained end-to-end under cross entropy loss. Class weight could potentially be incorporated in the cost function to reduce false negative rate.

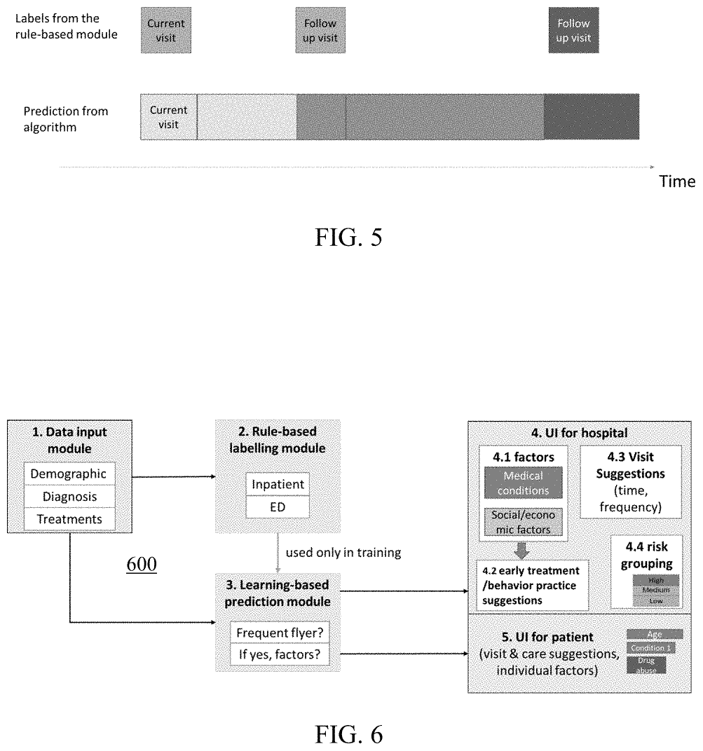

Referring to FIG. 5 is an output of the model. The direct output can be a prediction of whether a patient would be a superuser in the coming year. The one-year definition is the same as the definition, where one-year rolling window from the current admission. Consequently, once there is a new admission, the prediction algorithm is triggered with the information of the new admission and the prediction one-year window starting from one year from the new admission. The outputs are sequentially and updated with every new admission.

According to an embodiment, features can be scored at an individual level and/or at a group level. There are various feature scoring methods such as permutation test, Sharpley values, and attention plots. Sharpley values is a general game-theoretic approach to score feature importance of almost any algorithms such as logistic regression, tree-based methods, as well as deep learning methods. It scores the contribution of each feature based on the factor of its contribution within combinations including that feature over the combinations excluding that feature. Alternatively, attention plots can be created a learnable a soft attention mask during training neural networks, therefore there is no need for extra procedures.

Using one or a combination of the feature scoring methods, the system can score features on both group level as well as individual level. The group level gives top driving factors of frequent flyers such as medical condition factors and socioeconomic factors. This gives an indication of actionable efforts to help reduce future frequent readmission in hospitals. In our design, the result of group feature score is part of user interface for hospital staff. The individual level top driving factors could help an individual understand the reason for potentially frequent admissions. The result of this scoring is part of user interface for patient. One can also tracking the various of driving factors over time. According to an embodiment, from the predictive scores, the patients can be grouped to high risk, medium risk, and low risk by customized designed thresholds. Various color coding can be utilized for various risk groups.

Returning to method 100 in FIG. 1, at step 150 of the method, the system estimates using the trained model that whether the frequent readmission is due to a medical condition and/or due to a socioeconomic condition if the patient is determined in step 140 of the method to be a frequent readmission patient.

Alternatively, at step 160 of the method, the system predicts using the trained model a frequent readmission risk level if the patient is determined in step 140 of the method to be at risk of being a frequent readmission patient. The predicted frequent readmission risk level further comprises an identification of a medical factor and/or socioeconomic factor impacting the frequent readmission risk.

At step 170 of the method, the patient frequent readmission risk system generates, using the trained model, one or more recommendations based in least in part on the estimated medical and/or socioeconomic condition, and/or in part on the predicted frequent readmission risk level. According to an embodiment, if the patient's frequent readmission is estimated to be due to a medical condition or if the patient's risk of being a frequent readmission patient is based on a medical factor, the recommendation comprises a medical intervention such as a diagnosis, treatment, or other medical intervention. According to an embodiment, if the patient's frequent readmission is estimated to be due to a socioeconomic condition or if the patient's risk of being a frequent readmission patient is based on a socioeconomic factor, the recommendation comprises a socio-behavioral intervention such as in-facility and/or post-discharge social care, or other socio-behavioral intervention.

At step 180 of the method, the patient frequent readmission risk system displays or otherwise provides the generated recommendation to a clinician or other user via a user interface. The display may comprise information about the patient, the input data for the patient, whether the patient is a frequent readmission patient or is at risk of being a frequent readmission patient, and a medical condition and/or socioeconomic condition associated with or affecting the frequent readmission or frequent readmission risk, among other information. The recommendation and other information may be communicated by wired and/or wireless communication to another device. For example, the system may communicate the recommendation and other information to a mobile phone, computer, laptop, wearable device, and/or any other device configured to allow display and/or other communication of the recommendation and other information. The user interface can be any device or system that allows information to be conveyed and/or received, and may include a display, a mouse, and/or a keyboard for receiving user commands.

According to an embodiment, the user interface displays information to a clinician or other healthcare professional. For example, the display may include a group-level factor analysis submodule that provides one or more top factors that contribute to frequent visits. For instance, the factors may include medical factors like age, chronic disease, and socioeconomic factors such as lack of insurance, among many other factors. According to an embodiment, the display may include an early recommendation of treatment and behavioral improvement suggestions to modify or implement medical factors and social/economic factors respectively. According to an embodiment, the display may include a visit recommendation submodule that suggests a frequency of hospital visits, which potentially improves operational efficiency and clinical outcome. According to an embodiment, the display may include a risk-grouping submodule that groups patients into low risk, medium, and high risk of frequent visit based on prediction of frequent visit frequency.

According to an embodiment, the user interface displays information to a patient. For example, the display may include visit recommendations and help the patient understand the factor or factors that contribute to visit frequency. The user interface may also enable the patient to track their health status, among other information.

At optional 190 of the method, the clinician or other decisionmaker utilizes the displayed generated recommendation and/or other information about the patient in patient care decision-making. For example, if the patient's frequent readmission is estimated to be due to a medical condition or if the patient's risk of being a frequent readmission patient is based on a medical factor, the displayed recommendation comprises a medical intervention such as a diagnosis, treatment, or other medical intervention. The clinician or other decisionmaker can then implement the diagnosis, treatment, or other medical intervention in an attempt to prevent readmission, or to lower readmission frequency. As another example, if the patient's frequent readmission is estimated to be due to a socioeconomic condition or if the patient's risk of being a frequent readmission patient is based on a socioeconomic factor, the displayed recommendation comprises a socio-behavioral intervention such as in-facility and/or post-discharge social care, or other socio-behavioral intervention. The clinician or other decisionmaker can then implement the in-facility and/or post-discharge social care, or other socio-behavioral intervention, in an attempt to prevent readmission, or to lower readmission frequency. The decisionmaker can consider or implement decisions such as follow-up frequency and/or type for the patient, home visits, social care visits, monitoring at home, and/or many other decisions. These decisions, and their implementation, can be an attempt by the clinician to affect—and preferrable to lower—the readmission frequency of the patient. The possible or potential effect of the decision on the readmission frequency of the patient may be seen immediately or a period of time after the decision and/or implementation of the decision.

Referring to FIG. 2 is a schematic representation of a patient frequent readmission risk system 200. System 200 may be any of the systems described or otherwise envisioned herein, and may comprise any of the components described or otherwise envisioned herein. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, storage 260 of system 200 may store one or more algorithms, modules, and/or instructions to carry out one or more functions or steps of the methods described or otherwise envisioned herein. For example, the system may comprise, among other instructions or data, an electronic medical record system 270, training dataset 280, data processing instructions 262, training instructions 263, trained model 264, and/or reporting instructions 265.

According to an embodiment, the electronic medical record system 270 is an electronic medical records database from which the information about the patient, including the plurality of demographic and/or medical features about the patient, may be obtained or received. The electronic medical records database may be a local or remote database and is in communication the patient risk score analysis system 200. According to an embodiment, the patient frequent readmission risk system comprises an electronic medical record database or system 270 which is optionally in direct and/or indirect communication with system 200. In addition to the electronic medical record system 270, the information about the patient, including the plurality of demographic and/or medical features about the patient, may be obtained or received from other sources including those not shown in FIG. 2, such as home monitoring equipment, hospital systems, and other sources.

According to an embodiment, the training dataset 280 is a dataset that may be stored in a local or remote database and is in communication with the patient frequent readmission risk system 200. According to an embodiment, the patient frequent readmission risk system comprises a training data set 280. The training data can comprise medical information about a patient, including but not limited to demographics, previous inpatient and/or emergency department visits, physiological measurements such as vital data, physical observations, and/or diagnosis, among many other types of medical information.

According to an embodiment, the data processing instructions 262 direct the system to retrieve and process input data which is used to train the patient frequent readmission risk model 264. The data processing instructions 262 direct the system to for example, receive or retrieve input data or medical data to be used by the system as needed, such as from electronic medical record system 270 and/or training dataset 280, among many other possible sources. As described or otherwise envisioned herein, the input data can comprise a wide variety of input types from a wide variety of sources.

According to an embodiment, the data processing instructions 262 also direct the system to process the input data to generate a plurality of demographic and/or medical features for a plurality of patients, which are used to train the patient frequent readmission risk model. This can be accomplished by a variety of embodiments for feature identification, extraction, and/or processing. The outcome of the feature processing is a set of demographic and/or medical features related to readmission frequency analysis for a patient, which thus comprises a training data set that can be utilized to train the risk model 264.

According to an embodiment, the training instructions 263 direct the system to utilize the processed data to train the patient frequent readmission risk model 264. The model can be any machine learning algorithm, classifier, or model sufficient to utilize the type of input data provided, and to generate the analyses described or otherwise envisioned herein. Thus, the system comprises a trained patient frequent readmission risk model 264 configured to generate a patient frequent readmission recommendation for a patient, as described or otherwise envisioned herein.

According to an embodiment, the reporting instructions 265 direct the patient frequent readmission risk system to generate and provide the generated recommendation to a clinician or other user via a user interface. The recommendation may include information about the patient, the input data for the patient, whether the patient is a frequent readmission patient or is at risk of being a frequent readmission patient, and a medical condition and/or socioeconomic condition associated with or affecting the frequent readmission or frequent readmission risk, among other information. The recommendation and other information may be communicated by wired and/or wireless communication to another device. For example, the system may communicate the recommendation and other information to a mobile phone, computer, laptop, wearable device, and/or any other device configured to allow display and/or other communication of the recommendation and other information. The user interface can be any device or system that allows information to be conveyed and/or received, and may include a display, a mouse, and/or a keyboard for receiving user commands.

Referring to FIG. 6, in one embodiment, is a flowchart of a method 600 for determining and providing a recommendation generated by the patient frequent readmission risk system to a clinician and/or to a patient. The input for the system comprises information such as patient demographics, diagnosis, treatments, prior visit history, and/or other information. A rule-based model or module utilizes the patient information to identify the patient as a frequent readmission patient and/or to identify the patient as being at risk for being a frequent readmission patient. The system can also identify one or more medical and/or socioeconomic factors that influence the frequent readmissions or the risk of frequent readmission. The patient frequent readmission risk system can also generate recommendations to ameliorate or otherwise address the identified one or more medical and/or socioeconomic factors that influence the frequent readmissions or the risk of frequent readmission. The system provides the recommendations to a clinician or other user via a user interface. The system can also generate information for the patient based on the generated recommendations, and can provide the information to the patient via a user interface.

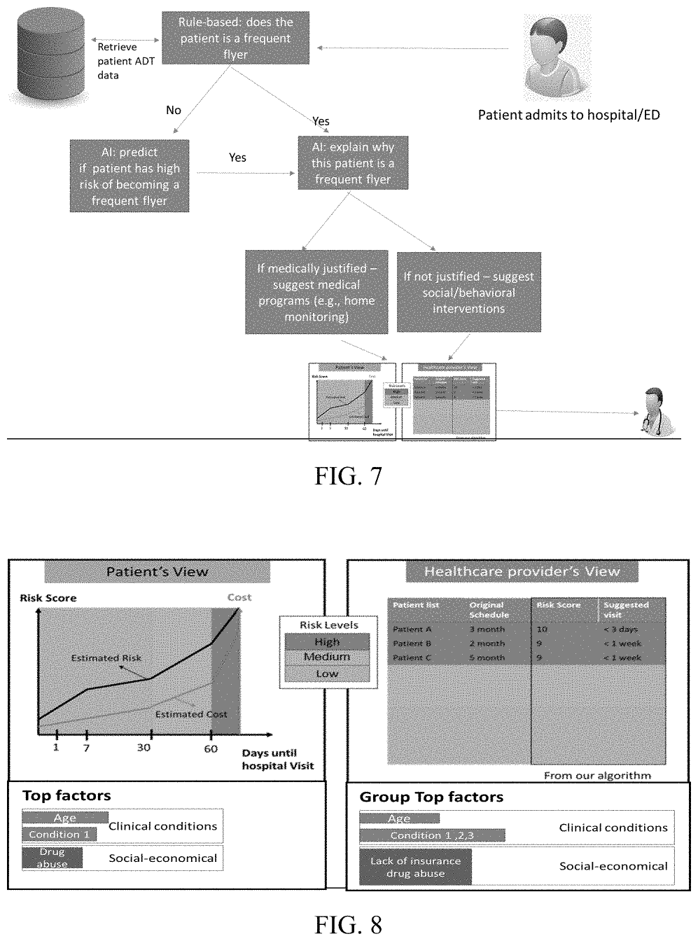

Referring to FIG. 7, in one embodiment, is a decision flow of recommendations or suggestions for a clinician and/or a user. Frequent visits due to medical conditions, predictive modeling provides early warning and anticipate early treatment suggestions and home-care recommendations. For frequent visits due to social/economic factors, the system can provide suggestions on programs such as suitable insurance plans, improvement of behavioral practices, and other recommendation. According to an embodiment, the system can automatically connect to a third-party database of community resources and can suggest relevant community resources and programs nearby. For patients that are superusers due to a medical condition, the system can recommend home monitoring programs that are relevant to the patient' specific medical condition, among other recommendations.

Referring to FIG. 8, in one embodiment, is a schematic representation of a display of a user interface 240 of patient frequent readmission risk system 200, comprising information generated and provided by the system. According to an embodiment, the user interface for the healthcare facility or clinician provides information on group-level factors and subgrouping based on risk, and other information. According to an embodiment, the user interface for the patient provides information such as suggestions of visits, treatment, care, insurance, behavioral practices, and other information. Colors and other indicators can be utilized to demonstrate a frequency or risk level. For example, green can indicate a low risk or frequency level, yellow can indicate an intermediate risk or frequency level, and red can indicate a high risk or frequency level.

According to an embodiment, the patient frequent readmission risk system is configured to process many thousands or millions of datapoints in the input data used to train the model, as well as to process and analyze the plurality of patient features. For example, generating a functional and skilled trained classifier or model using an automated process such as feature identification and extraction and subsequent training requires processing of millions of datapoints from input data and the generated features. This can require millions or billions of calculations to generate a novel trained classifier or model from those millions of datapoints and millions or billions of calculations. As a result, each trained classifier or model is novel and distinct based on the input data and parameters of the machine learning algorithm, and thus improves the functioning of the patient frequent readmission risk system. Thus, generating a functional and skilled trained classifier or model comprises a process with a volume of calculation and analysis that a human brain cannot accomplish in a lifetime, or multiple lifetimes.

In addition, the patient frequent readmission risk system can be configured to continually receive patient features, perform the analysis, and provide periodic or continual updates via the user interface. This requires the analysis of thousands or millions of datapoints on a continual basis to optimize the reporting, requiring a volume of calculation and analysis that a human brain cannot accomplish in a lifetime.

By providing an improved patient frequency readmission risk analysis, this novel patient frequent readmission risk system has an enormous positive effect on patient frequency readmission risk analysis compared to prior art systems. As just one example in a clinical setting, by providing a system that can improve patient frequency readmission risk analysis and generate ameliorating recommendations, the system can facilitate treatment decisions and improve survival outcomes, thereby leading to saved lives. The system can also reduce the visits and resources utilized by superusers, thereby leading to millions of dollars in saved expenses.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method for generating and presenting a patient frequent readmission recommendation using a patient frequent readmission risk system, comprising:
receiving, at the patient frequent readmission risk system, patient information, wherein the patient information comprises a plurality of demographic and/or medical features;
extracting, by a processor of the patient frequent readmission risk system, the plurality of demographic and/or medical features from the received information;
analyzing, by a trained model of the patient frequent readmission risk system, the extracted demographic and/or medical features to determine whether the patient is a frequent readmission patient or is at risk of being a frequent readmission patient;
estimating, if the patient is determined to be a frequent readmission patient, whether the frequent readmission is due to a medical condition and/or due to a socioeconomic condition, or predicting a frequent readmission risk level if the patient is determined to be at risk of being a frequent readmission patient, wherein the predicted frequent readmission risk level further comprises an identification of a medical factor and/or socioeconomic factor impacting the frequent readmission risk;
generating a recommendation based at least in part on the estimated condition or the frequent readmission risk level, wherein the recommendation comprises a medical intervention if the frequent readmission is estimated to be due to a medical condition or if the risk of being a frequent readmission patient is based on a medical factor, and wherein the recommendation comprises a socio-behavioral intervention if the frequent readmission is estimated to be due to a socioeconomic condition or if the risk of being a frequent readmission patient is based on a socioeconomic factor; and
providing the recommendation via a user interface of the patient frequent readmission risk system.

2. The method of claim 1, further comprising the step of implementing the provided medical intervention or socio-behavioral intervention.

3. The method of claim 1, wherein the provided recommendation comprises a visualization of the patient's visitation frequency, frequent readmission risk level, recommendation, medical condition, and/or socioeconomic condition.

4. The method of claim 1, wherein at least some of the patient information is received from a patient home monitoring device.

5. The method of claim 1, wherein at least some of the patient information is received from an electronic medical records database or system.

6. The method of claim 1, wherein the medical features comprise information about patient diagnosis, patient treatment, and/or or a number of healthcare facility admissions during a previous predetermined time period.

7. The method of claim 1, wherein a patient is determined to be a frequent readmission patient if the patient has been admitted to a healthcare facility at least a predetermined number of times during a previous predetermined time period.

8. The method of claim 1, further comprising the step of training the model of the patient frequent readmission risk system using historical patient data.

9. A patient frequent readmission risk system configured to generate and present a patient frequent readmission recommendation, comprising:
a trained patient frequent readmission risk model configured to determine, based on one or more demographic and/or medical features, whether a patient is a frequent readmission patient or is at risk of being a frequent readmission patient;
a processor configured to: (i) receive patient information comprising a plurality of demographic and/or medical features; (ii) extract the plurality of demographic and/or medical features from the received information; (iii) analyze, by the trained patient frequent readmission risk model, the extracted demographic and/or medical features to determine whether the patient is a frequent readmission patient or is at risk of being a frequent readmission patient; (iv) estimate, if the patient is determined to be a frequent readmission patient, whether the frequent readmission is due to a medical condition and/or due to a socioeconomic condition, or predict a frequent readmission risk level if the patient is determined to be at risk of being a frequent readmission patient, wherein the predicted frequent readmission risk level further comprises an identification of a medical factor and/or socioeconomic factor impacting the frequent readmission risk; (v) generate a recommendation based at least in part on the estimated condition or the frequent readmission risk level, wherein the recommendation comprises a medical intervention if the frequent readmission is estimated to be due to a medical condition or if the risk of being a frequent readmission patient is based on a medical factor, and wherein the recommendation comprises a socio-behavioral intervention if the frequent readmission is estimated to be due to a socioeconomic condition or if the risk of being a frequent readmission patient is based on a socioeconomic factor; and
a user interface configured to present to a user the generated recommendation.

10. The patient frequent readmission risk system of claim 9, wherein the processor is further configured to receive an input from a user regarding an implementation of the provided medical intervention or socio-behavioral intervention.

11. The patient frequent readmission risk system of claim 9, wherein the provided recommendation comprises a visualization of the patient's visitation frequency, frequent readmission risk level, recommendation, medical condition, and/or socioeconomic condition.

12. The patient frequent readmission risk system of claim 9, wherein at least some of the patient information is received from a patient home monitoring device.

13. The patient frequent readmission risk system of claim 9, wherein at least some of the patient information is received from an electronic medical records database or system of the patient frequent readmission risk system.

14. The patient frequent readmission risk system, wherein a patient is determined to be a frequent readmission patient if the patient has been admitted to a healthcare facility at least a predetermined number of times during a previous predetermined time period.

15. The patient frequent readmission risk system, wherein the processor is further configured to train the patient frequent readmission risk model using historical patient data.
